(19) United States
(12) Patent Application Publication
(10) Pub. No.: US 2026/0224313 A1
(43) Pub. Date: Aug. 6, 2026

(54) CANNULA ASSEMBLY FOR ENABLING AUTOMATED TASKS DURING A ROBOTIC SURGICAL PROCEDURE

(71) Applicant: NEW VIEW SURGICAL, Boston, MA (US)

(72) Inventors: Bryce C. Klontz, JR., Boston, MA (US); Joseph Peter Corrigan, Hardwick, Cambridgeshire (GB); Joshua John Gibson, Needingworth, Cambridgeshire (GB); Rachel Mary Rakvica, Newbury, MA (US)

(73) Assignee: New View Medical, Inc., Newton Center, MA (US)

(21) Appl. No.: 19/157,421

(22) PCT Filed: Mar. 7, 2024
(86) PCT No.: PCT/US2024/018786

§ 371 Date: Aug. 18, 2025

Related U.S. Application Data
(60) Provisional application No. 63/489,483, filed on Mar. 10, 2023.

(51) Int. Cl. A61B 34/32; A61B 34/30; A61B 90/00

(52) U.S. Cl. CPC A61B 34/32; A61B 90/37; A61B 2034/301

(57) ABSTRACT

A robotic surgical system for performing a robotic surgical procedure on a patient. The system includes a cannula assembly including a cannula tube having a distal end portion configured for insertion into a patient. The cannula tube may also have a housing coupled to the cannula tube so as to be positioned within the patient when the distal end of the cannula tube is inserted into the patient. The housing may be movable relative to the cannula tube between a closed position and an open position, the housing including an image sensor configured to provide image data of the surgical site when the housing is in the open position within the patient. The system may also include at least one robotic surgical tool configured to perform a surgical task during the robotic surgical procedure. The system may also include a processor configured to determine, based at least in part upon the image data of the surgical site, that the surgical task is safe to be performed by the robotic surgical tool in an automated mode.

### CROSS-REFERENCE TO RELATED APPLICATIONS

This application claims the benefit of U.S. Provisional Application Ser. No. 63/489,483, filed Mar. 10, 2023, the complete disclosure of which is incorporated herein by reference for all purposes.

### BACKGROUND

Minimally invasive surgery involves making small incisions into a body of a patient to insert surgical tools. Increasingly, minimally invasive surgeries are being performed robotically, via robotic surgical systems. For example, a surgeon may perform a robotic laparoscopic procedure using multiple cannulas inserted through individual incisions that accommodate various robotic surgical tools, including illumination devices and imaging devices. To accomplish the insertion, cannula assemblies may be used to puncture the body cavity. A cannula assembly often includes an obturator and a cannula. An obturator is a device placed inside a cannula, the obturator having either a sharp tip (e.g., a pointed cutting blade) or a blunt tip for creating an incision or opening in the patient for the cannula to pass through. After the obturator and cannula are inserted, the obturator is removed, leaving the cannula in place for use in inserting the robotic surgical tools into the surgical space within a patient. Typically, in addition to cannulas forming individual incisions for robotic surgical tools, an individual incision may also be made through the patient by a cannula that is thereafter dedicated to holding an illumination and/or imaging device, e.g., a traditional endoscope or laparoscope. A surgical tool combining a cannula and an imaging device in a single unit is disclosed, for example, in U.S. Pat. No. 8,834,358, the disclosure of which is herein incorporated by reference in its entirety.

### SUMMARY

The following presents a simplified summary of the claimed subject matter in order to provide a basic understanding of some aspects of the claimed subject matter. This summary is not an extensive overview of the claimed subject matter. It is intended to neither identify key or critical elements of the claimed subject matter nor delineate the scope of the claimed subject matter. Its sole purpose is to present some concepts of the claimed subject matter in a simplified form as a prelude to the more detailed description that is presented later.

In accordance with various embodiments thereof, there is provided a robotic surgical system for performing a robotic surgical procedure on a patient. The system includes a cannula assembly including a cannula tube having a distal end portion configured for insertion into a patient. The cannula tube may also have a housing coupled to the cannula tube so as to be positioned within the patient when the distal end of the cannula tube is inserted into the patient. The housing may be movable relative to the cannula tube between a closed position and an open position, the housing including an image sensor configured to provide image data of the surgical site when the housing is in the open position within the patient. The system may also include at least one robotic surgical tool configured to perform a surgical task during the robotic surgical procedure. The system may also include a processor configured to determine, based at least in part upon the image data of the surgical site, that the surgical task is safe to be performed by the robotic surgical tool in an automated mode.

The robotic surgical system may also include, in various embodiments, an automated control mechanism configured to communicate with the robotic surgical tool actuation mechanism to control the operation of the robotic surgical tool. In some embodiments, upon the processor determining that the surgical task is safe to be performed by the robotic surgical tool in an automated mode, the processor may be configured to transmit a signal to the robotic surgical tool actuation mechanism that instructs the robotic surgical tool actuation mechanism to receive its operation instructions for actuating the robotic surgical tool from the automated control mechanism. The robotic surgical may also include a manual control mechanism configured to enable a user to manually operate the robotic surgical tool via the robotic surgical tool actuation mechanism.

In embodiments, the robotic surgical system may also include a mode selection interface enabling the user to select either a manual mode or the automated mode. The processor may be configured to make the determination that the surgical task is safe to be performed by the robotic surgical tool in an automated mode by processing stored safety data related to at least one of the robotic surgical tool, the surgical procedure, and/or the patient. In some embodiments, the user can only select the automated mode via the mode selection interface after the processor determines, based upon processing the safety data, that the surgical task is safe to be performed by the robotic surgical tool in the automated mode.

In various embodiments, the robotic surgical system is configured such that the surgical task that the processor determines to be safe to be performed by the robotic surgical tool is the task of moving the robotic surgical tool from a current position to a desired position. In further embodiments, the robotic surgical system is configured such that the surgical task that the processor determines to be safe to be performed by the robotic surgical tool is the task of changing the robotic surgical tool from a current operational state to a desired operational state.

Still further, the robotic surgical system may also include, in some embodiments, a second imaging device configured for insertion into a patient and to provide second image data of the patient's anatomy. In such an embodiment, the processor may be configured to receive the first and second image data and to process the first and second image data so as to generate combined image data of the patient's anatomy. The robotic surgical system may also include a display device to display the combined image to a user. Additionally or alternatively, the robotic surgical system may also include a spatial information processor configured to determine the spatial relationship between the patient's anatomy and the robotic surgical tool.

In still further embodiments, there is described hereinbelow a robotic surgical system for performing a robotic surgical procedure on a patient that includes a cannula assembly. The cannula assembly may include a cannula tube having a longitudinal axis, a proximal end portion and a distal end portion configured for insertion into a patient. The cannula tube may also have a housing coupled to the cannula tube between the proximal and distal ends of the cannula tube so as to be positioned within the patient when the distal end of the cannula tube is inserted into the patient. The housing may be movable relative to the cannula tube between a closed position and an open position. The housing may include a light source and an image sensor configured to provide first image data of the patient's anatomy when the housing is in the open position within the patient. The robotic surgical system may also include a second imaging device configured for insertion into a patient and to provide second image data of the patient's anatomy.

According to this embodiment, the robotic surgical system may also include an image processor configured to receive the first and second image data and to process the first and second image data so as to generate combined image data of the patient's anatomy. The robotic surgical system may also include at least one robotic surgical tool configured to perform a surgical task during the robotic surgical procedure. Still further, the robotic surgical system may also include a processor configured to determine, based at least in part on the combined image, that the surgical task is suitable to be performed by the robotic surgical tool in an automated mode. In some embodiments, the robotic surgical system may also include an automated control mechanism configured to control the operation of the robotic surgical tool.

Upon the processor determining that the surgical task is suitable to be performed by the robotic surgical tool in an automated mode, the second processor may be configured to transmit a signal for the automated control mechanism to actuate the robotic surgical tool. The robotic surgical system may also include a manual control mechanism configured to enable a user to manually operate the robotic surgical tool. Still further, the robotic surgical system may also include a mode selection interface enabling the user to select either a manual mode or the automated mode. The processor may be configured to make the determination that the surgical task is suitable to be performed by the robotic surgical tool in an automated mode by processing safety data related to the operation of the robotic surgical tool. The robotic surgical system may be configured such that the user can only select the automated mode via the mode selection interface after the second processor determines, based upon processing the safety data, that the surgical task is safe to be performed by the robotic surgical tool in the automated mode. In some embodiments, the surgical task may be moving the robotic surgical tool from a current position to a desired position. Additionally or alternatively, the surgical task may be changing the robotic surgical tool from a current operational state to a desired operational state.

In an embodiment, the robotic surgical system may also include a display device to display the combined image to a user. Still further, the robotic surgical system may also include a spatial information processor configured to determine the spatial relationship between the patient's anatomy and the robotic surgical tool. According to various embodiments, the processor may be incorporated into the cannula assembly, or may be external to the cannula assembly. Additional or alternatively, the processor(s) may include various controller devices, some of which are internal relative to the cannula assembly and some of which are external relative to the cannula assembly, these various controller devices operating to perform, either separately or together, the various operations described herein. Of course, it will be recognized that, multiple different processors may be employed to perform the various operations, there being no limit on the number or configuration of processors that may be employed.

Among various other advantages provided by certain embodiments as will be evident from the Detailed Description below, there may also be the benefit that fewer punctures through a patient, e.g., through an abdominal wall or other bodily surface, are made during a robotic surgical procedure. As set forth above, a surgeon typically performs a laparoscopic procedure using multiple cannulas inserted through individual incisions, wherein at least one such cannula and incision is occupied by an illumination/imaging device, such as a traditional endoscope and/or laparoscope. According to various embodiments, there may be provided a cannula assembly and/or system therefor that eliminates the need for this separate puncture by a cannula assembly for an endoscope/laparoscope, since it provides, in certain embodiments, a cannula assembly which provides both an illumination/imaging device (e.g., mounted or coupled to the cannula tube) and an internal lumen through which a separate robotic surgical tool (e.g., a surgical stapler, etc.) may be inserted. The reduction of at least one puncture during a robotic surgical procedure, as may be enabled in certain embodiments, may improve the safety of the robotic surgical procedure by avoiding potential complications, reducing pain and/or speeding the patient's recovery.

### BRIEF DESCRIPTION OF THE DRAWINGS

FIG. 1 is a system block diagram that illustrates cannula assemblies employed in a robotic surgical procedure, in accordance with various embodiments.

FIG. 2 shows a block diagram illustrating an example of a device controller in accordance with various embodiments.

FIG. 3 shows a block diagram illustrating an example of an imaging/navigation controller for a system in accordance with embodiments.

### DETAILED DESCRIPTION

Generally, there is provided systems and methods related to robotic surgical systems and, more particularly, to imaging systems for a robotic surgical system. In various embodiments, and as will be set forth in detail below, there are provided cannula assemblies for use in a robotic surgical system that provides selective, automated control of a robotic surgical tool real during a robotic surgical procedure which, in some embodiments, enables a user to selectively choose between manual and/or automated tool control when a processor determines, based on continuously updated imaging data, that it is safe for an automated mode to be selected.

Reference will now be made in detail to specific embodiments illustrated in the accompanying drawings. In the following detailed description, numerous specific details are set forth to provide a thorough understanding thereof. However, it will be apparent to one of ordinary skill in the art that embodiments may be practiced without these specific details. In other instances, known methods, procedures, components, circuits, and networks have not been described in detail so as not to unnecessarily obscure aspects of the embodiments.

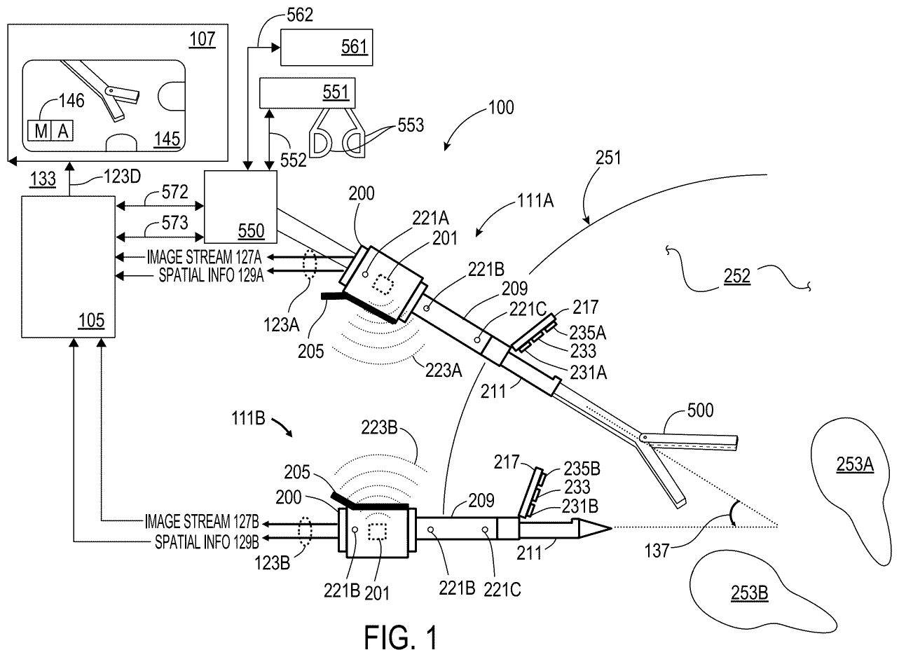

FIG. 1 shows a robotic surgical system 100 illustrating an example embodiment. In this embodiment, there is shown a robotic surgical system 100 in which there are two cannula assemblies 111A, 111B. Although FIG. 1 illustrates two such cannula assemblies, it should be understood that certain advantages may be obtained with a single such cannula assembly. This embodiment having two cannula assemblies will have additional advantages as shown and described below.

In the embodiment shown in FIG. 1, each of the cannula assemblies 111A, 111B include a housing 200, a device controller 201, an actuator handle 205, a cannula tube 209, an obturator 211, and a sensor housing 217. Although not shown herein, it should be understood by those skilled in the art that the cannula assembly 111A (and other cannula assemblies shown and described herein) may include other components and features in addition to those described herein. For example, any of the herein-described cannula assemblies 111A, 111B may include sealing components, such as an instrument seal for sealing around a robotic surgical tool or instrument inserted therethrough, a zero seal for sealing the cannula assembly in the absence of any tool or instrument inserted therethrough, and/or any number of different ports, e.g., insufflation or irrigation ports, for the introduction of various gases or liquids into the surgical site.

The cannula tubes 209 may be formed of a variety of cross-sectional shapes. For example, the cannula tubes 209 can have a generally round or cylindrical, ellipsoidal, triangular, square, rectangular, and D-shaped (in which one side is flat). The cannula tube 209 may include an internal lumen 202 into which the obturator 211 is inserted. The obturator 211 can be retractable and/or removable from the cannula tube 209. In some embodiments, the obturator 211 is made of solid, non-transparent material. In another embodiment, all or parts of the obturator 211 are made of optically transparent or transmissive material such that the obturator 211 does not obstruct the view through the camera (discussed below). The obturator 211 may have a tip shape that is configured to penetrate, either via incision or via insertion between tissue planes, through the abdominal wall 251 of the patient.

The sensor housing 217 can be integral with the cannula tube 209 or it may be formed as a separate component that is coupled to the cannula tube 209. In either case, the sensor housing 217 can be disposed on or coupled to the cannula tube 209 at a position proximal to the distalmost end of the cannula tube 209 such that it is positioned within the patient's body when the distal end portion of the cannula tube 209 has been inserted into the patient. In some embodiments, the sensor housing 217 can be actuated by the actuator handle 205 to open, for example, after being inserted into the patient's 117 body cavity 252. The sensor housing 217 can reside along cannula tube 209 in the distal direction such that it is positioned within the body cavity 252 of a patient (e.g., patient 117) during a robotic surgical procedure. At the same time, the sensor housing 217 can be positioned proximal to the distal end such that it does not interfere with the insertion of the distal end of the cannula tube 209 as it is inserted into a patient (e.g., patient 117).

In some embodiments, each of the sensor housings 217 may include one or more image sensors 231A, 231B and a light source 235A, 235B. The light sources 235A, 235B may be dimmable light-emitting device, such as a LED, a halogen bulb, an incandescent bulb, or other suitable light emitter. The image sensors 231A, 231B may be devices configured to detect light reflected from the light source 235 and output an image signal. The image sensors 231A, 231B can be, for example, a charged coupled device (“CCD”) or other suitable imaging sensor. In some embodiments, the image sensors 231A, 231B includes at least two lenses providing stereo imaging. In some embodiments, the image sensors 231A, 231B can be an omnidirectional camera. The image data based on image signals generated by image device 231A can eventually be overlaid onto the image data based on image signals generated by image device 231B, or vice versa, so as to provide a combined image stream, as will be described more fully below.

The cannula tube 209, the obturator 211, and the sensor housing 217 of the individual cannula assemblies 111A, 111B can be inserted into the body cavity 252 of a patient (e.g., patient 117) and positioned relative to each other, e.g., such as at an angle 137 with respect to each other, so as to provide differing fields-of-view from the sensor housing 217 of robotic surgical tools, e.g., surgical stapler 500, and patient anatomical features, e.g., patient anatomical features 253A, 253B, within the body cavity 252 of the patient 117, as will be described in additional detail below.

The device controller 201 may be one or more devices that process signals and data to generate respective image streams 127A, 127B and spatial information 129A, 129B of the cannula assemblies 111A, 111B. Spatial data, e.g., spatial information 129A, 129B, is data that relates to the relative position of various components, in this case the cannula assembles 111A, 111B and the patient anatomical features 253A, 253B. Various different spatial data components are contemplated. For example, in the embodiment shown, the cannula assemblies 111A, 111B may include spatial data components, e.g., in the form of antennas 221A, 221B, 221C. In some embodiments, the device controller 201 can determine the spatial information 129A, 129B by processing data from spatial sensors (e.g., accelerometers) to determine the relative position, angle, and rotation of the cannula assemblies 111A, 111B. In some embodiments, the device controller 201 can also determine the spatial information 129A, 129B by processing range information received from sensors (e.g., image sensor 231 and LiDAR device 233) in the sensor housing 217. Additionally, in some embodiments, the device controller 201 can process the spatial information 129A, 129B by processing signals received via the antennas 221A, 221B, 221C to determine relative distances of the cannula assemblies 111A, 111B. It is understood that, in some embodiments, less than all, e.g., only one or none, of the cannula assemblies 111A, 111B provides spatial information 129. However, the spatial information, as shown and described herein, may be advantageous to ensure that image streams are accurately combined relative to each other.

As mentioned above, in some embodiments, the sensor housing 217 can include a LiDAR device 233. The LiDAR device 233 can include one or more devices that illuminate a region with light beams, such as lasers, and determine distance by measuring reflected light with a photosensor. The distance can be determined based on a time difference between the transmission of the beam and detection of backscattered light. For example, using the LiDAR device 233, the device controller 201 can determine spatial information 129 by sensing the relative distance and rotation of the cannulas 209 or the sensor housing 217 inside a body cavity.

Additionally, where antennas are employed, the antennas 221A, 221B, 221C can be disposed along the long axis of the cannula assemblies 111A, 111B. In some embodiments, the antennas 221A, 221B, 221C can be placed in a substantially straight line on one or more sides of the cannula assemblies 111A, 111B. For example, two or more lines of the antennas 221A, 221B, 221C can be located on opposing sides of the housing 203 and the cannula tube 209. Although FIG. 1 shows a single line of the antennas 221A, 221B, 221C on one side of the cannula assemblies 111A, 111B, it is understood that the additional lines of the antennas 221A, 221B, 221C can be placed in opposing halves, thirds, or quadrants of the cannula assemblies 111A, 111B.

As illustrated in FIG. 1, in some embodiments, the device controllers 201 can transmit a ranging signal 223. In some embodiments, the location signals are ultra-wideband (“UWB”) radio signal usable to determine a distance between the cannula assemblies 111A, 111B less than or equal to 1 centimeter based on signal phase and amplitude of the radio signals, as described in IEEE 802.15.4Z. The device controller 201 can determine the distances between the cannula assemblies 111A, 111B based on the different arrival times of the ranging signals 223A and 223B at their respective antennas 221A, 221B, 221C. For example, referring to FIG. 1, the ranging signal 223A emitted by cannula assembly 111A can be received by cannula assembly 111B at antenna 221C and an amount of time (T) after arriving at antenna 221B. By making a comparison of the varying times of arrival of the ranging signal 223A at two or more of the antennas 221A, 221B, 221C, the device controller 201 of cannula assembly 111B can determine its distance and angle from cannula assembly 111A. It is understood that the transmitters can be placed at various suitable locations within the cannula assemblies 111A, 111B. For example, in some embodiment, the transmitters can be located in the cannulas 209 or in the sensor housings 217.

In some embodiments, the spatial sensors 317 can include one or more of, piezoelectric sensors, mechanical sensors (e.g., a microelectronic mechanical system (“MEMS”), or other suitable sensors for detecting the location, velocity, acceleration, and rotation of the cannula assemblies (e.g., cannula assemblies 111A, 111B).

As set forth above, FIG. 1 also illustrates a robotic surgical tool, e.g., in this case a robotic surgical stapler 500. Of course, it should be recognized that the robotic surgical tool 500 may be any conceivable type of robotic surgical tool, depending on the robotic surgical procedure being performed, and that the description herein of a surgical stapler is merely exemplary of the type of surgical tools that may be employed. Robotic surgical tool 500 is actuatable by a robotic surgical tool actuation mechanism 550, which may include any combination of drive mechanisms, motors, gears, controllers, etc. that is configured to actuate the robotic surgical tool 500.

The robotic surgical tool actuation mechanism 550 is connected to, and controllable by, a manual control mechanism 551. The manual control mechanism 551 may be a typical robotic console having, e.g., handles 553 that are manipulatable by a user to control the actuation of the robotic surgical tool 500 (and in some cases, multiple robotic surgical tools). The manual control mechanism 551 may send and receive manual control signals 552 to and from the robotic surgical tool actuation mechanism 550. The robotic surgical tool actuation mechanism 550 then uses those manual control signals 552 to actuate its drive mechanisms, motors, gears, controllers, etc. in accordance with the operations and functions inputted by the user, e.g., in the case of a robotic surgical stapler, e.g., to physically move the robotic surgical tool 500 to a different location, to clamp the stapler jaws closed, to fire the stapling mechanism so as to fasten the clamped tissue, etc. Of course, it should be recognized by those skilled in the art that, because the robotic surgical tool 500 may be any conceivable type of robotic surgical tool, the specific operations and functions that may be inputted by the user via manual control mechanism 551 and that may thereby be actuated by the robotic surgical tool actuation mechanism 550 are not limited in any way.

The robotic surgical tool actuation mechanism 550 may also be connected to, and controllable by, an automated control mechanism 561. The automated control mechanism 561 may store various programs and/or instructions that relate to the operation and function of the robotic surgical tool 500. Advantageously, the automated control mechanism 561 may be any type of processor or controller that is configured to send and receive automated control signals 562 to and from the robotic surgical tool actuation mechanism 550. The robotic surgical tool actuation mechanism 550 may then use those automated control signals 562 to actuate its drive mechanisms, motors, gears, controllers, etc. in accordance with the operations and functions pre-programmed into its stored programs and/or instructions, e.g., one or more of the same operations and/or functions set forth above in connection with the manual control mechanism 551 such as physically moving the robotic surgical tool 500 to a different location, and/or, in the case of a surgical stapler, clamping and/or firing the stapling mechanism, or any other conceivable type of operation or function of a robotic surgical tool.

The robotic surgical tool actuation mechanism 550 may also be connected to the imaging/navigation controller 105. In some embodiments, if it is determined, e.g., by the imaging/navigation controller 105 based on the various image data received thereby, that it is safe for the robotic surgical tool 500 to be actuated for a specific surgical task without user input, e.g., without the user needing to manually control the performance of the specific surgical task, the imaging/navigation controller 105 may send signals, e.g., automated tool actuation signal 573, that indicates to the robotic surgical tool actuation mechanism 550 that the automated control mechanism 561 controls of actuating the surgical robotic tool 500.

In other embodiments, the robotic surgical tool actuation mechanism 550 may be configured to transmit and receive other signals from the image/navigation controller 105. For example, in one such embodiment, the robotic surgical tool actuation mechanism 550 may be configured to transmit and receive a mode selection signal 572 from the image/navigation controller 105. The mode selection signal 572 may be a signal that is generated by a user input. Specifically, in accordance with this embodiment, if it is determined, e.g., by the imaging/navigation controller 105 based on the processing of the various image data received thereby, that it is safe for the robotic surgical tool 500 to be actuated for a specific surgical task without user input, e.g., without the user needing to manually control the performance of the specific surgical task, the imaging/navigation controller 105 may enable the user to make a mode selection via an input/output interface (not shown, but may be any selection mechanism provided by, e.g., a graphic user interface or GUI). The selection that the user may make via the input/output interface may be to either manually actuate the robotic surgical tool 500 to perform the surgical task, e.g., via the manual control mechanism 551, or to have the surgical task performed automatically by the robotic surgical tool 500, e.g., via the automated control mechanism 561. This selection by the user between a manual actuation of the robotic surgical tool 500 and an automated actuation thereof may provide an additional level of user discretion that may provide the user with an increased level of control over the operations and functions of the robotic surgical tool actuation mechanism 550.

Additionally, and as also set forth above, FIG. 1. illustrates patient anatomical features, e.g., patent anatomical features 253A, 253B. Again, it should be recognized that the patent anatomical features 253A, 253B may be any conceivable anatomical features, depending on the location of the body at which the robotic surgical procedure is being performed.

As set forth above, the cannula assemblies 111A, 111B may include a processor or device controller 201 that is configured to receive the image data from the image sensors 231A, 231B and to perform certain processing steps regarding the image data prior to its being displayed on a separate display device, e.g., display device 107 having a display 145. The processor or device controller 201 can be a computing device connecting the cannula assemblies 111A, 111B to the display 107, e.g., either directly (or indirectly via additional processors), through one or more wired or wireless communication channels 123A, 123B. In the embodiment shown in FIG. 1, the system 100 also includes the imaging/navigation controller 105, that performs additional processing steps, as will be described in further detail below. The imaging/navigation controller 105 may also be a computing device that is connected to the display device 107 and the cannula assembly 111A through the one or more wired or wireless communication channels 123A, 123B.

The communication channels 123A, 123B may use various serial, parallel, video transmission protocols suitable for their respective signals such as image streams 127A, 127B and processed image stream 133. The imaging/navigation controller 105 can include hardware, software, or a combination thereof for performing operations. The display device 107 can be a liquid crystal display (LCD) display, organic light emitting diode displays (OLED), cathode ray tube display, or other suitable display device. In some embodiments, the display device 107 can be a stereoscopic head-mounted display, such as a virtual reality headset.

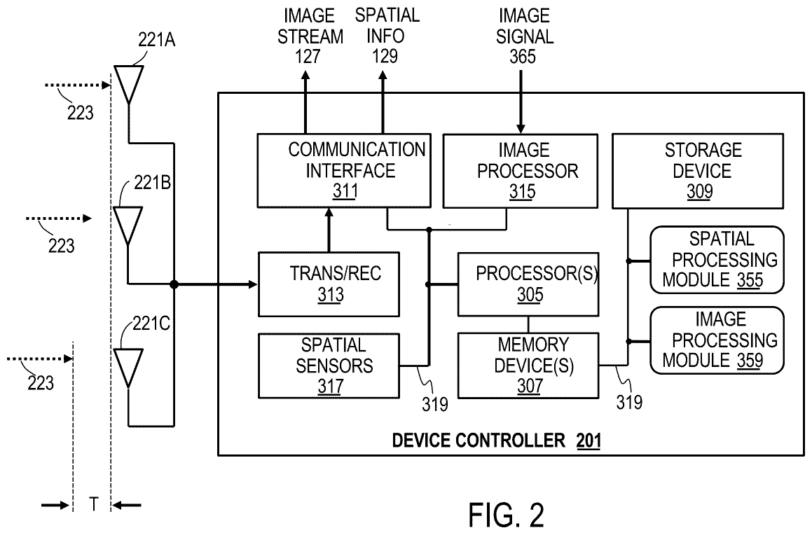

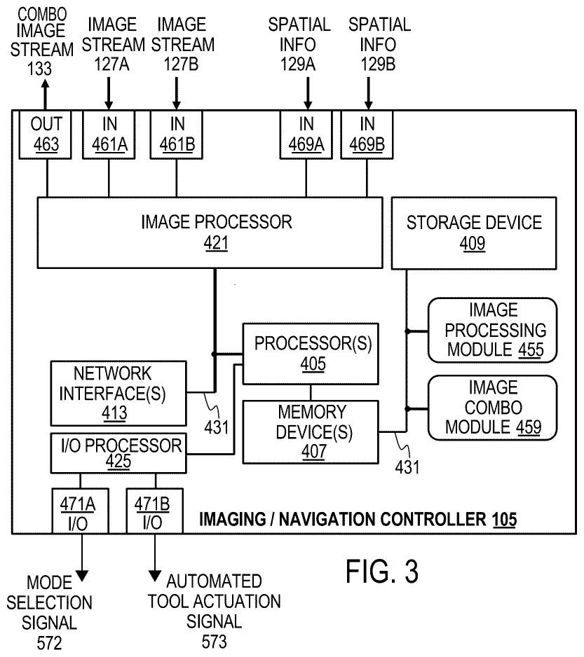

It should be noted that, while the description hereinbelow describes various components, operations and functions as potentially being present and/or performed by one or more of the device controller 201, the image/navigation controller 105, the automated control mechanism 561 and/or the manual control mechanism 551, it is contemplated that the below-described components, operations and functions may be present or performed entirely in a single one of these devices, that additional controllers/processor may be present that perform any one or more or portions of said operations or functions, and/or that the components described herein may be shared across these devices (and/or such additional processors) such that the device controller 201, the image/navigation controller 105, the automated control mechanism 561 and the manual control mechanism 551 share responsibility for performing any one or more of the herein-described operations or functions. As will be shown below, FIGS. 2 and 3 illustrate an embodiment in which device controller 201 has components for, and performs, certain operations and functions, while the image/navigation controller 105 has components for, and performs, certain operations and functions. It should be recognized by those skilled in the art that, in accordance with other embodiments thereof, there may be included processors, either internal or external to the cannula assemblies 111A, 111B, for performing these operations and functions, and that, although described in connection with a certain processor, there is no intent herein to be limited to any particular structure or location of such components, operations or functions. The example embodiment described hereinbelow is merely one way that such processors may be employed.

In operation, and in accordance with an example embodiment as mentioned above, the image sensors 231A, 231B generate image signals relating to the body cavity 252 of the patient, including image signals relating to, e.g., robotic surgical tool 500 and patient anatomical features 253A, 253B. These image signals are processed by the device controller 201 to generate respective image streams 127A, 127B relating thereto.

Simultaneously, any one or more of the spatial data devices, e.g., antennas 221A, 221B, 221C or LiDAR device 233, etc., may generate spatial data relating to the cannula assemblies 111A, 111B. This spatial data may be received by and processed by the device controller 201 to generate respective spatial information 129A, 129B relating thereto. The image data streams 127A, 127B and/or the spatial information 129A, 129B may then be used, e.g., via the device controller 201 and/or the image/navigation controller 105, to generate stereoscopic image data of the surgical site, e.g., including the robotic surgical tool 500 and the patient anatomical features 253A, 253B within the body cavity 252. Advantageously, the stereoscopic image data, e.g., of the robotic surgical tool 500 and the patient anatomical features 253A, 253B, may be utilized by the system 100 to provide a 3D display to the surgeon on the display device 107.

In addition to using the stereoscopic image data of the surgical site, e.g., of the robotic surgical tool 500 and the patient anatomical features 253A, 253B, to provide a 3D display to the surgeon on the display device 107, the imaging/navigation controller 105 may also use the stereoscopic image data of the robotic surgical tool 500 and the patient anatomical features 253A, 253B to generate current tool condition data of the robotic surgical tool 500. This current tool condition data may be data that relates to the current position, e.g., current spatial information, of the robotic surgical tool 500 and/or the patient anatomical features 253A, 253B. Specifically, the stereoscopic image data of the robotic surgical tool 500 and the patient anatomical features 253A, 253B may enable the imaging/navigational processor 105 to calculate current positional data that represents where the robotic surgical tool 500 and the patient anatomical features 253A, 253B are currently located within the body cavity 252. Additionally or alternatively, the current tool condition data may be data that relates to the current operational state of the robotic surgical tool 500, e.g., data that relates to a surgical stapler being in an unclamped or unfired operational state. The current tool condition data may relate, in various embodiments, to any current state of the robotic surgical tool 500.

Still further, the imaging/navigation controller 105 may then be employed to determine desired tool condition data of the robotic surgical tool 500. The desired tool condition data of the robotic surgical tool 500 may be data that relates to any desired state of the robotic surgical tool 500. For example, in some embodiments, the desired tool condition data of the robotic surgical tool 500 may be data that relates to a desired engagement position of the robotic surgical tool relative to the patient anatomical features, e.g., a position at which the robotic surgical tool 500 is desired to be engaged with the patient anatomical features 253A, 253B for the purpose of conducting its intended surgical task. By way of example, referring to the surgical stapler 500 depicted in FIG. 1, the desired engagement position may be the position at which the surgical stapler 500 will be in position to engage with patient tissue 253.

Additionally or alternatively, the desired tool condition data may be data that relates to the desired operational state of the robotic surgical tool 500, e.g., data that relates to a surgical stapler fully actuated so as to be in a clamped or fired operational state. Of course, it should be understood by persons of skill in the art that the desired tool condition data may be any conceivable type of data depending on the type of the robotic surgical tool being used in the robotic surgical procedure, the particular type of patient tissue to be engaged during the robotic surgical procedure, the particular surgical task desired to be performed, the unique physical characteristics of the patient, etc. The imaging/navigation processor 105 may be configured to receive, whether via imaging sensors 231A, 231B or via user inputs (described in additional below in connection with, e.g., the I/O processors 425 in FIG. 3) or via stored data memory locations (also described in additional detail below in connection with, e.g., the storage device 409 in FIG. 3), such data about any or all of these factors and may utilize such data in determining the desired tool condition data.

Still further, the imaging/navigation controller 105 may then be employed to compare the current tool condition data of the robotic surgical tool 500 to the desired tool condition data of the robotic surgical tool 500 and to generate proposed tool actuation data. More specifically, the imaging/navigation controller 105 may use the current tool condition data and the desired tool condition data of the robotic surgical tool 500 to generate, e.g., data that relates to how to move the robotic surgical tool 500 from its current state to its desired state. For example, in some embodiments, where the robotic surgical tool 500 is desired to be moved from a first position to a second position within the patient, the proposed tool actuation data may relate to a proposed navigational path via which the robotic surgical tool 500 may be moved between its current position relative to a patient anatomical feature 253A, and its desired engagement position relative to the patient anatomical feature 253A. Continuing with the example described hereinabove, the imaging/navigation controller 105 may use data relating to the current and desired positions of the robotic surgical tool 500 relative to the patient anatomical feature 253A to generate a navigational path along which the robotic surgical tool 500 may be manipulated in order for the robotic surgical tool 500 to be moved from its current position relative to a patient anatomical feature 253A to its desired engagement position relative to the patient anatomical feature 253A.

Additionally or alternatively, where it is desired that the robotic surgical tool 500 change its operational state (rather than its physical location), the proposed tool actuation data may relate to, e.g., data that would actuate a surgical stapler from a current unclamped operation state to a desired clamped operational state, or that actuates said surgical stapler from a staples-unfired operational state to staples-fired operational state. As mentioned above, it should be understood by persons of skill in the art that the proposed tool actuation data may refer to any conceivable data depending on the type of the robotic surgical tool being used in the robotic surgical procedure, the particular type of patient tissue to be engaged during the robotic surgical procedure, the particular surgical task desired to be performed, the unique physical characteristics of the patient, etc. And, as above, the imaging/navigation processor 105 may be configured to utilize any or all of such types of data in determining the proposed tool actuation data.

In still further embodiments, it is also contemplated that the imaging/navigation controller 105 may compare the proposed tool actuation data to stored safety data to determine whether the proposed tool actuation data is safe to be performed. The stored safety data may be any type of stored data that relates to the safety of the robotic surgical procedure being performed. For example, the stored safety data may consist of safety data relating to the particular robotic surgical tool 500 being employed, e.g., in the case of a surgical stapler, the stored safety data may relate to optimal clamping angles for the surgical stapler, preferred stapler lengths or staple configurations, or any other conceivable safety information that would be useful for a surgeon to know as the robotic surgical procedure is being conducted. Other types of stored safety data may also be employed, e.g., the stored safety data may consist of safety data relating to the patient's anatomy, such as preferred tissue thickness ranges across which a surgical stapler can be fired, or may consist of data related to known anatomical structures, e.g., vasculature or major arteries, that should not be stapled across, or may consist of anatomical feature data that is patient-specific. Of course, any other conceivable safety information that would be useful for a surgeon to know as the robotic surgical procedure is being conducted, may be employed in various embodiments.

If the imaging/navigation controller 105 determines that it is safe for the surgeon to move the robotic surgical tool 500 between its current condition, e.g., its current position and/or operational state, and the desired condition, e.g., its desired position and/or operational state, via the proposed tool actuation data, the imaging/navigation controller 105 may generate the automated tool actuation signal 573 and transmit same to the robotic surgical tool actuation mechanism 550. In this way, the automated tool actuation signal 573 may enable the robotic surgical tool actuation mechanism 550 to automatically receive its actuation instructions from the automated control mechanism 561 for the purpose of conducting the desired surgical task with the robotic surgical tool 500.

Additionally or alternatively, if the imaging/navigation controller 105 determines that it is safe for the surgeon to move the robotic surgical tool 500 between its current condition, e.g., its current position and/or operational state, and the desired condition, e.g., its desired position and/or operational state, via the proposed tool actuation data, the imaging/navigation controller 105 may generate a mode selection field 146 which is, e.g., displayed to a user on display device 107. The mode selection field 146 may be employed by a user to select the mode, e.g., selectable by a user via an input/output device and which may be either a manual mode or an automated mode, by which the user prefers the robotic surgical tool 500 be actuated. In embodiments, the mode selection field 146 may be in the form of any user input device and may be displayed in any way, e.g., as a selector switch, dial, button, etc., or any display or mechanism that enables a user to input a selection.

In some embodiments, the generation and display of the mode selection field 146 lets a user know that the proposed tool actuation is safe for the robotic surgical tool 500 to be so actuated. For example, the mode selection field 146 may remain hidden from view, e.g., so that only manual operation is possible, unless and until the imaging/navigation controller 105 determines that it is safe for the surgeon to move the robotic surgical tool 500 between its current condition and the desired condition. In other embodiments, the mode selection field 146 may be displayed to a user but may be disabled, e.g., unable to be selected by the user, unless and until the imaging/navigation controller 105 determines that it is safe for the surgeon to move the robotic surgical tool 500 between its current condition and the desired condition. Still further, the mode selection field 146 may include any text, symbols or messages that convey to the user that the imaging/navigation controller 105 has determined, or has not determined, that it is safe for the surgeon to move the robotic surgical tool 500 between its current condition and the desired condition.

In various embodiments thereof, it is contemplated that the various different operation steps, such as those described hereinabove, can be performed numerous times over the course of a robotic surgical procedure. Optimally, for example, it is contemplated that the various different operation steps described hereinabove can be performed continuously over the course of a robotic surgical procedure, allowing the various operational steps to be continuously re-evaluated and/or adjusted during the robotic surgical procedure. In such an embodiment, the image streams 127A, 127B can generate constantly-updated image data relating to the surgical site, e.g., the robotic surgical tool 500 and the patient anatomical features 253A, 253B. This may thereby allow the processors, e.g., one or more of the device controller 201, the imaging/navigation controller 105, the automated control mechanism 561 and/or the manual control mechanism 551, to constantly update throughout the surgical procedure the current tool condition data and the desired tool condition data relating to the robotic surgical tool 500 and the patient anatomical features 253A, 253B. In this way, the processors can take into account the movement of the robotic surgical tool 500 and/or the movement of the patient anatomical features 253A, 253B, during the robotic surgical procedure so as to continuously adjust, if needed, the proposed tool actuation data generated therefrom, e.g., to adjust the proposed tool actuation data to move the surgical stapler along a different navigational path if the position of the tissue that it is aiming towards moves during the course of the surgical procedure. Similarly, the processors can continuously take into account changes in the operational state of the robotic surgical tool 500 and/or changes that the user may desire to implement in the operational state of the robotic surgical tool 500 during the robotic surgical procedure so as to continuously adjust, if needed, the proposed tool actuation data generated therefrom, e.g., if the user doesn't like the position on which the stapler is being clamped on the patient's tissue and wishes to unclamp the surgical stapler jaws and reclamp it onto a different section of tissue, and/or if the system is configured to continuously display the progress of the stapler firing mechanism so that the user can visually track that the staples are correctly and sequentially being fired into the tissue for the purpose of determining a secure and predictable fastener line. Of course, it will be recognized by persons of skill in the art that, since there are no limitations to the type of robotic surgical tool 500 that may be employed herein, and that there is no limitations to the type of surgical procedure that may be performed, that likewise there is no limitation to the different ways that the proposed tool actuation data may be updated during the course of the surgical procedure. Such an arrangement, e.g., wherein the various different operation steps described hereinabove are performed continuously over the course of a robotic surgical procedure so as to allow continuous adjustments to the proposed tool actuation data during the robotic surgical procedure, provides significant advantages over robotic surgical systems that lack such functionality, because the systems and methods described herein may enable the actuation of the robotic surgical tools to not only be operated in an automated mode, if desired, but also provide a user with real-time data that allows the user to adjust the course of the surgical procedure if so desired.

FIG. 2 shows a functional block diagram illustrating an example of a device controller 201 in accordance with aspects. It is noted, as mentioned above, that the device controller 201 shown and described herein is merely representative of various possible equivalent-computing devices that can perform the processes and functions described herein. To this extent, in some embodiments, the functionality provided by the device controller 201 can be any combination of general and/or specific purpose hardware and/or program instructions. In each embodiment, the program instructions and hardware can be created using standard programming and engineering techniques. In the embodiment shown, the device controller 201 may include a processor 305, a memory device 307, a storage device 309, a communication interface 311, a transmitter/receiver 313, an image processor 315, spatial sensors 317, and a data bus 319.

In various embodiments, the processor 305 may include one or more microprocessors, microchips, or application-specific integrated circuits. The memory device 307 may include one or more types of random-access memory (RAM), read-only memory (ROM) and cache memory employed during execution of program instructions. The processor 305 may use the data buses 319 to communicate with the memory device 307, the storage device 309, the communication interface 311, the image processor 315, and the spatial sensors 317. The storage device 309 may comprise a computer-readable, non-volatile hardware storage device that stores information and program instructions. For example, the storage device 309 can be one or more, flash drives and/or hard disk drives. The transmitter/receiver 313 can be one or more devices that encodes/decodes data into wireless signals, such as the ranging signal 223.

The processor 305 executes program instructions (e.g., an operating system and/or application programs), which can be stored in the memory device 307 and/or the storage device 309. The processor 305 may also execute program instructions of a spatial processing module 355 and an image processing module 359. The spatial processing module 335 can include program instructions that determine the spatial information 129 by combining spatial data provided from the transmitter/receiver 313 and the spatial sensors 317. The image processing module 359 can include program instructions that, using the image signals 365 from the imaging sensors 231A, 231B register and overlay the images to generate the image streams 127A, 127B. The image processor 315 can be a device configured to receive an image signal 365 from an image sensor (e.g., image sensors 231A, 231B) and condition images included in the image signal 365. In accordance with aspects, conditioning the image signal 365 can include normalizing the size, exposure, and brightness of the images. Also, conditioning the image signal 365 can include removing visual artifacts and stabilizing the images to reduce blurring due to motion. Additionally, the image processing module 359 can identify and characterize structures in the images.

FIG. 3 shows a functional block diagram illustrating an imaging/navigation controller 105 in accordance with aspects thereof. It is noted, as mentioned above, that the imaging/navigation controller 105 shown and described herein is merely representative of various possible equivalent-computing devices that can perform the processes and functions described herein. To this extent, in some embodiments, the functionality provided by the imaging/navigation controller 105 can be any combination of general and/or specific purpose hardware and/or program instructions, and the program instructions and hardware can be created using standard programming and engineering techniques. The imaging/navigation controller 105 may include, e.g., a processor 405, a memory device 407, a storage device 409, a network interface 413, an image processor 421, an I/O processor 425, and a data bus 431. Also, the imaging/navigation controller 105 can include input connections 461A, 461B for connecting the image streams 127A, 127B, respectively, to the image processor 421. In addition, the imaging/navigation controller 105 may also include input connections 469A, 469B for connecting the spatial information streams 129A, 129B, respectively, to the image processor 421. Further, the imaging/navigation controller 105 may include output connection 463 for transmitting the combined image stream 133 from the image processor 421 to, e.g., a display device such as display device 107. Still further, the imaging and navigation controller 105 may also include input/output connections 471A, 471B that receive/transmit data signals, e.g., the mode selection signal 572 and/or the automated tool actuation signal 573, respectively, to and from the I/O processor 425.

In embodiments, the imaging/navigation controller 105 can include one or more microprocessors, microchips, or application-specific integrated circuits. The memory device 407 can include one or more types of random-access memory (RAM), read-only memory (ROM) and cache memory employed during execution of program instructions. Additionally, the imaging/navigation controller 105 can include one or more data buses 431 by which its processor(s) 405 communicates with the memory device 407, the storage device 409, the network interface 413, the image processor 421, and the I/O processor 425. The storage device 409 can comprise a computer-readable, non-volatile hardware storage device that stores information and program instructions. For example, the storage device 409 can be one or more, flash drives and/or hard disk drives. The storage device 409 may store any type of useful data. For example, in various embodiments, it may store, as set forth previously, data relating to the type of the robotic surgical tool being used in the robotic surgical procedure, the particular type of patient tissue to be engaged during the robotic surgical procedure, the particular surgical task desired to be performed, the unique physical characteristics of the patient, etc.

Still further, the storage device 409 may store data relating to safety data. For example, as set forth previously, such safety data may be any type of data that the processor 405 may employ to help determine whether it is safe for the particular robotic surgical tool 500 to move between its current tool condition, e.g., its current position and/or its current operational state, and its desired tool condition, e.g., the desired tool position and/or its desired operational state. Still further, the safety data may be any safety data relating to the robotic surgical tool 500 being employed (e.g., in the case of a surgical stapler, safety data relating to optimal clamping angles for the surgical stapler, preferred stapler lengths or staple configurations, etc.), safety data relating to the patient's anatomy (e.g., such as preferred tissue thickness ranges across which a surgical stapler can be fired, data related to known anatomical structures like vasculature or major arteries that should not be stapled across, etc.) or any other conceivable type of safety information.

The I/O processor 425 can be connected the processor 405 and can include or be connected to any device that enables an individual to interact with the processor 405 (e.g., a user interface) and/or any device that enables the processor 405 to communicate with one or more other computing devices using any type of communications link. For example, the I/O processor 425 may include data relating to the mode selection signal 572. As set forth above, if the imaging/navigation controller 105 determines that it is safe for the surgeon to move the robotic surgical tool 500 between its current condition, e.g., its current position or operational state, and the desired condition, e.g., its desired position or operational state, via the proposed tool actuation data, the imaging/navigation controller 105 may generate a mode selection field 146 which is, e.g., displayed to a user on display device 107. The mode selection field 146 may be generated by and/or connected to I/O processor 425 such that a user may select the mode, e.g., either a manual mode or an automated mode, by which the user prefers the robotic surgical tool 500 be actuated, and the robotic surgical tool actuation mechanism 550 may be configured to receive a mode selection signal 572. Similarly, if the imaging/navigation controller 105 determines that it is safe for the surgeon to move the robotic surgical tool 500 between its current condition, e.g., its current position or operational state, and the desired condition, e.g., its desired position or operational state, via the proposed tool actuation data, the imaging/navigation controller 105 may generate the automated tool actuation signal 573 and transmit same to the robotic surgical tool actuation mechanism 550. In this way, the automated tool actuation signal 573 may enable the robotic surgical tool actuation mechanism 550 to automatically receive its actuation instructions from the automated control mechanism 561 for the purpose of conducting the desired surgical task with the robotic surgical tool 500.

Still further, the I/O processor 425 may include any type of device that enables a surgeon to input information useful to the robotic surgical procedure. For example, it may allow to be inputted information relating to, e.g., the type of the robotic surgical tool being used in the robotic surgical procedure, the particular type of patient tissue to be engaged during the robotic surgical procedure, the particular surgical task desired to be performed, the unique physical characteristics of the patient, safety data relating to the particular robotic surgical tool 500 being employed, safety data relating to the patient's anatomy, or any other conceivable type of safety information. In various embodiments, the I/O processor 425 can generate and receive, for example, digital and analog inputs/outputs according to various data transmission protocols.

The processor 405 executes program instructions (e.g., an operating system and/or application programs), which can be stored in the memory device 407 and/or the storage device 409. For example, the processor 405 may be employed, in various embodiments, to generate the herein above-referenced current condition data, e.g., data related to a current position of the robotic surgical tool 500 and/or data related to the current operational state of the robotic surgical tool 500. More specifically, and as set forth above, the processor 405 may use the stereoscopic image data of the robotic surgical tool 500 and the patient anatomical features 253A, 253B to generate current tool condition data that represents where the robotic surgical tool 500 and the patient anatomical features 253A, 253B are currently located within the body cavity 252 and/or the current operational state, e.g., clamped or unclamped, fired or unfired, etc., of the robotic surgical tool 500.

In still further embodiments, the processor 405 may be employed to, as set forth above, determine desired tool condition data, e.g., a position at which the robotic surgical tool 500 will be engaged with the patient anatomical features 253A, 253B for the purpose of conducting its intended surgical task and/or a desired operational state of the robotic surgical tool 500. The processor 405 may be configured to generate this desired tool condition data by processing other data (e.g., the type of robotic surgical procedure, the type of robotic surgical tool being used etc.) received from one or more different data sources (e.g., from the imaging sensors 231A, 231B, from the user inputs of the I/O processors, and/or from stored data memory locations such as the storage device 409). The processor 405 may also be employed to, as set forth above, generate proposed tool actuation data, e.g., a navigational path for moving the robotic surgical tool 500 to its desired engagement position relative to the patient anatomical features 253A, 253B and/or a set of instructions for moving the robotic surgical tool 500 from its current operational state to a desired operational state. Still further, the processor 405 may also be employed to, as previously described, compare the proposed tool actuation data to stored safety data (e.g., stored safety data that may be stored, for example, in storage device 409) to determine whether the proposed tool actuation data is safe for the surgeon to move the robotic surgical tool 500 between current and desired positions/states. In addition, the processor 405 may be employed, as described hereinabove, to generate or otherwise process the mode selection signal 572 and/or the automated tool actuation signal 573 so as to thereby communicate with the tool actuation mechanism 550.

In various embodiments, and as set forth above, the processor 405 may be configured to perform these different operation steps numerous times, and optimally to perform them continuously, over the course of the robotic surgical procedure. In this way, the processor 405 obtains and processes image data from the image sensors 231A, 231B in real-time, enabling the proposed tool actuation data generated by the processor 405 to be continuously updated to reflect changes in the relative positions or operational states of the robotic surgical tools 500 and the patient anatomical features 253A, 253B. In this way, the processor(s) can take into account the movement/operation of the robotic surgical tool 500 and/or the movement of the patient anatomical features 253A, 253B, during the robotic surgical procedure so as to adjust, if needed, the actuation instructions made thereby.

The processor 405 can also execute program instructions of an image processing module 455 and an image combination module 459. The image processing module 455 can be configured to stabilize the images to reduce the blurring, compensate for differences in tilt and rotation, remove reflections and other visual artifacts from the images, and normalize the images. Additionally, the image processing module 455 can be configured to identify and characterize structures, such as robotic surgical tools 500 and/or tissues 253A, 253B, in the images. Further, the imaging processing module 455 can be configured to determine obstructions in the overlapping fields of view and process the images streams 127A, 127B to remove the obstructions, if desirable.

The image combination module 459 can be configured to analyze images received in image streams 127A, 127B from the cannula assemblies and overlay them into a single, combined image stream 133 based on the spatial information. In some embodiments, the image combination module 459 generates the combined image stream 133 by registering and overlaying the image stream 127A, 127B based on the respective fields-of-view of the cannula assemblies. In some embodiments, either of the cannula assemblies can be selected by an operator (e.g., via I/O processor 425, additional connections thereto not shown in this example) as a primary cannula assembly (e.g., cannula assembly 111A), and the image combination module 459 can generate the combined image stream 133 by using the image stream 127B of the secondary cannula assembly to augment the image stream 127A. The combined image stream 133 can also provide a 3D view from the perspective of the primary cannula assembly (or vice versa). In some embodiments, the combined image stream 133 lacks certain obstructions removed by the image processing module 455.

The image processing module 421 may also, in accordance with various embodiments, operate to generate and display, e.g., the mode selection field 146. As set forth above, the mode selection field 146 may be any type of symbol or text that lets the surgeon know that the proposed tool actuation data is safe for the surgeon to implement and/or enables the user to select whether to implement a manual mode or an automated mode of operation. The image processing module 421 may generate the corresponding symbol, e.g., button, switch, etc., and provide data relating thereto to the image combination module 459 so that the mode selection field 146 may be accurately combined into the combined image stream 133 along with the other image streams 127A, 127B for display on the display device 107.

The systems and methods described hereinabove are not to be limited in terms of the particular embodiments described in this application, which are intended as illustrations of various aspects. Many modifications and variations can be made without departing from its spirit and scope, as will be apparent to those skilled in the art. Functionally equivalent methods and apparatuses, in addition to those enumerated herein, will be apparent to those skilled in the art from the foregoing descriptions. Such modifications and variations are intended to fall within the scope of the appended claims. Only the terms of the appended claims are intended to be limiting, along with the full scope of equivalents to which such claims are entitled. It is also to be understood that the terminology used herein, e.g., “and”, “or”, “including”, “at least” as well as the use of plural or singular forms, etc., is for the purpose of describing examples of embodiments and is not intended to be limiting.

1. A robotic surgical system for performing a robotic surgical procedure on a patient, comprising:
a cannula assembly including a cannula tube having a longitudinal axis, a proximal end portion and a distal end portion configured for insertion into a patient, the cannula tube having a housing coupled to the cannula tube between the proximal and distal ends of the cannula tube so as to be positioned within the patient when the distal end of the cannula tube is inserted into the patient, the housing movable relative to the cannula tube between a closed position and an open position, the housing including an image sensor configured to provide image data of the surgical site when the housing is in the open position within the patient;
at least one robotic surgical tool configured to perform a surgical task during the robotic surgical procedure; and
a processor configured to determine, based at least in part upon the image data of the surgical site, that the surgical task is safe to be performed by the robotic surgical tool in an automated mode.

2. The robotic surgical system of claim 1, further comprising:
an automated control mechanism configured to communicate with robotic surgical tool actuation mechanism to control the operation of the robotic surgical tool.

3. The robotic surgical system of claim 2, wherein, upon the processor determining that the surgical task is safe to be performed by the robotic surgical tool in an automated mode, the processor is configured to transmit a signal to the robotic surgical tool actuation mechanism that instructs the robotic surgical tool actuation mechanism to receive its operation instructions for actuating the robotic surgical tool from the automated control mechanism.

4. The robotic surgical system of claim 3, further comprising:
a manual control mechanism configured to enable a user to manually operate the robotic surgical tool via the robotic surgical tool actuation mechanism.

5. The robotic surgical system of claim 4, further comprising:
a mode selection interface enabling the user to select either a manual mode or the automated mode.

6. The robotic surgical system of claim 5, wherein the processor is configured to make the determination that the surgical task is safe to be performed by the robotic surgical tool in an automated mode by processing stored safety data related to at least one of the robotic surgical tool, the surgical procedure, and/or the patient.

7. The robotic surgical system of claim 6, wherein the user can only select the automated mode via the mode selection interface after the processor determines, based upon processing the safety data, that the surgical task is safe to be performed by the robotic surgical tool in the automated mode.

8. The robotic surgical system of claim 7, wherein the surgical task is moving the robotic surgical tool from a current position to a desired position.

9. The robotic surgical system of claim 7, wherein the surgical task is changing the robotic surgical tool from a current operational state to a desired operational state.

10. The robotic surgical system of claim 1, further comprising:
a second imaging device configured for insertion into a patient and to provide second image data of the patient's anatomy;
the processor configured to receive the first and second image data and to process the first and second image data so as to generate combined image data of the patient's anatomy;
a display device to display the combined image to a user.

11. (canceled)

12. The robotic surgical system of claim 1, further comprising:
a spatial information processor configured to determine the spatial relationship between an anatomy of the patient and the robotic surgical tool.

13. A robotic surgical system for performing a robotic surgical procedure on a patient, comprising:
a cannula assembly including a cannula tube having a longitudinal axis, a proximal end portion and a distal end portion configured for insertion into a patient, the cannula tube having a housing coupled to the cannula tube between the proximal and distal ends of the cannula tube so as to be positioned within the patient when the distal end of the cannula tube is inserted into the patient, the housing movable relative to the cannula tube between a closed position and an open position, the housing including a light source and an image sensor configured to provide first image data of the patient's anatomy when the housing is in the open position within the patient;
a second imaging device configured for insertion into a patient and to provide second image data of the patient's anatomy;
an image processor configured to receive the first and second image data and to process the first and second image data so as to generate combined image data of the patient's anatomy;
at least one robotic surgical tool configured to perform a surgical task during the robotic surgical procedure; and
a processor configured to determine, based at least in part on the combined image, that the surgical task is suitable to be performed by the robotic surgical tool in an automated mode.

14. The robotic surgical system of claim 13, further comprising:
an automated control mechanism configured to control operation of the robotic surgical tool.

15. The robotic surgical system of claim 14, wherein, upon the processor determining that the surgical task is suitable to be performed by the robotic surgical tool in an automated mode, the second processor is configured to transmit a signal for the automated control mechanism to actuate the robotic surgical tool.

16. The robotic surgical system of claim 15, further comprising:
a manual control mechanism configured to enable a user to manually operate the robotic surgical tool.

17. The robotic surgical system of claim 16, further comprising:
a mode selection interface enabling the user to select either a manual mode or the automated mode.

18. The robotic surgical system of claim 17, wherein the processor is configured to make the determination that the surgical task is suitable to be performed by the robotic surgical tool in an automated mode by processing safety data related to the operation of the robotic surgical tool.

19. The robotic surgical system of claim 18, wherein the user can only select the automated mode via the mode selection interface after the second processor determines, based upon processing the safety data, that the surgical task is safe to be performed by the robotic surgical tool in the automated mode.

20. The robotic surgical system of claim 19, wherein the surgical task is one of moving the robotic surgical tool from a current position to a desired position, or changing the robotic surgical tool from a current operational state to a desired operational state.

21. (canceled)

22. The robotic surgical system of claim 13, further comprising:
a spatial information processor configured to determine the spatial relationship between the patient's anatomy and the robotic surgical tool.
